(19) United States
(12) Patent Application Publication
(10) Pub. No.: US 2006/0093623 A1
(43) Pub. Date: May 4, 2006

(54) Subtype-matched inactivated whole virus vaccines for treating patients with HIV infection

(75) Inventors: Jean Marie Andrieu, Paris (FR); Louis Wei-Lu, Paris (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: BIOVAXIM LIMITED, a corporation of United Kingdom, London (GB)

(21) Appl. No.: 11/243,094

(22) Filed: Oct. 4, 2005

Related U.S. Application Data
(60) Provisional application No. 60/615,729, filed on Oct. 4, 2004.

(51) Int. Cl. A61K 39/21

(52) U.S. Cl. 424/208.1

(57) ABSTRACT

Inactivated whole HIV of a particular subtype is used for vaccines and pharmaceutical compositions containing the vaccines. The vaccines can be used to treat individuals chronically infected with HIV, by inducing a protective cellular immune response in the individuals against the same HIV subtype used to produce the vaccine.

### RELATED APPLICATION

This patent application claims the benefit of U.S. Provisional Application No. 60/615,729, filed Oct. 4, 2004. This earlier provisional application is hereby incorporated by reference.

### FIELD OF THE INVENTION

This invention relates to vaccines for the treatment of viral infection, in particular, for the treatment of HIV infection in humans.

### BACKGROUND

Twenty years after the discovery of human immunodeficiency virus (HIV), it has been difficult to provide an effective preventive or therapeutic vaccine. Recent projections from the World Health Organization and the Joint United Nations Program on HIV/AIDS indicate that if the pandemic progresses at its current rate, there may be 45 million new infections by 2010 (J. Stover et al., Can we reverse the HIV/AIDS pandemic with an expanded response? Lancet 360, 73-7 (2002)). Although significant progress has been achieved in extending the survival of HIV-infected people and reducing maternal-newborn HIV transmission by antiretroviral therapy, there are an increasing number of patients who develop drug resistance and/or severe drug-related adverse effects under long-term antiretroviral therapy. Additional alternative therapeutic strategies are thus urgently needed to protect HIV-infected individuals from disease progression.

In a large-scale human phase III trial, one candidate vaccine aimed at eliciting humoral immunity to neutralize HIV has recently failed to demonstrate its efficacy (J. Cohen, HIV/AIDS. Vaccine results lose significance under scrutiny. Science 299, 1495 (2003)). This lack of protection is in keeping with the difficulties of current vaccine designs to elicit effective neutralizing antibodies (Nab) against HIV in vivo (I. K. Srivastava, J. B. Ulmer & S. W. Barnett, Neutralizing antibody responses to HIV: role in protective immunity and challenges for vaccine design. Expert Rev Vaccines 3 Suppl 1, S33-52 (2004)). Such an inability to raise efficient Nab is probably determined by the nature of the infectious agent. For example, there is so far no successfully preventative vaccine for chronic infections such as tuberculosis, leprosy, and hepatitis C virus infection. In contrast, successful vaccines prevent acute infectious diseases such as polio, measles, diphtheria, tetanus, or small pox through the induction of Nab that can be also transferred transplacentally or via milk to protect foetus or newborn from infections.

Long-lasting cellular immunity can potentially control disease in situations where the infectious agent is not eradicated, such as in chronic infections with tuberculosis, leprosy, hepatitis B or C virus, and HIV. In this regard, vigorous virus-specific CD4⁺ T-helper type 1 (Th1)-cell and effector (perforin⁺) cytotoxic T lymphocytes (CTL) responses were shown to be associated with control of viremia and long-term non-progression in individuals with chronic HIV-1 infection (E. S. Rosenberg et al., Vigorous HIV-1-specific CD4+ T cell responses associated with control of viremia. Science 278, 1447-50 (1997); C. J. Pitcher et al., HIV-1-specific CD4+ T cells are detectable in most individuals with active HIV-1 infection, but decline with prolonged viral suppression. Nat Med 5, 518-25 (1999); J. J. Zaunders et al., Identification of circulating antigen-specific CD4+ T lymphocytes with a CCR5+, cytotoxic phenotype in an HIV-1 long-term non-progressor and in CMV infection. Blood (2003); M. J. Boaz, A. Waters, S. Murad, P. J. Easterbrook & A. Vyakarnam, Presence of HIV-1 Gag-specific IFN-gamma+IL-2+ and CD28+IL-2+ CD4 T cell responses is associated with nonprogression in HIV-1 infection. J Immunol 169, 6376-85 (2002); S. A. Younes et al., HIV-1 viremia prevents the establishment of interleukin 2-producing HIV-specific memory CD4+ T cells endowed with proliferative capacity. J Exp Med 198, 1909-22 (2003); A. Harari, S. Petitpierre, F. Vallelian & G. Pantaleo, Skewed representation of functionally distinct populations of virus-specific CD4 T cells in HIV-1-infected subjects with progressive disease: changes after antiretroviral therapy. Blood 103, 966-72 (2004); and C. Hess et al., HIV-1 specific CD8+ T cells with an effector phenotype and control of viral replication. Lancet 363, 863-6 (2004)). In addition, early intervention with highly active antiretroviral therapy (HAART) during or shortly after acute infection was associated with enhanced HIV-1-specific CD4⁺ Th1-cell responses (U. Malhotra et al., Effect of combination antiretroviral therapy on T-cell immunity in acute human immunodeficiency virus type 1 infection. J Infect Dis 181, 121-31 (2000) and A. Oxenius et al., Early highly active antiretroviral therapy for acute HIV-1 infection preserves immune function of CD8+ and CD4+ T lymphocytes. Proc Natl Acad Sci USA 97, 3382-7 (2000)). In contrast, at a later stage, HAART led to the decline of HIV-1-specific CD4⁺ Th1-cell and CTL responses (C. J. Pitcher et al., HIV-1-specific CD4+ T cells are detectable in most individuals with active HIV-1 infection, but decline with prolonged viral suppression. Nat Med 5, 518-25 (1999); C. M. Gray et al., Frequency of class I HLA-restricted anti-HIV CD8+ T cells in individuals receiving highly active antiretroviral therapy (HAART). J Immunol 162, 1780-8 (1999); and S. A. Kalams et al., Levels of human immunodeficiency virus type 1-specific cytotoxic T-lymphocyte effector and memory responses decline after suppression of viremia with highly active antiretroviral therapy. J Viral 73, 6721-8 (1999)), suggesting that the functional capacities of HIV-1-capturing antigen presentation cells (APCs) (which are required for the induction of the immune response) are progressively lost along the course of the infection (D. Mcllroy et al., Low CD83, but normal MHC class II and costimulatory molecule expression, on spleen dendritic cells from HIV+ patients. AIDS Res Hum Retroviruses 14, 505-13 (1998); F. Grassi et al., Depletion in blood CD11c-positive dendritic cells from HIV-infected patients. Aids 13, 759-66 (1999); H. Donaghy et al., Loss of blood CD11c(+) myeloid and CD11c(−) plasmacytoid dendritic cells in patients with HIV-1 infection correlates with HIV-1 RNA virus load. Blood 98, 2574-6 (2001); and J. Pacanowski et al., Reduced blood CD123+ (lymphoid) and CD11c+ (myeloid) dendritic cell numbers in primary HIV-1 infection. Blood 98, 3016-21 (2001)). In this context, a therapeutic vaccine aimed at eliciting HIV-specific long-lasting cellular immune responses could be feasible under the two prerequisite conditions: 1) the discovery of an appropriate immunogen capable of eliciting a strong, broad, and sustained protective cellular immunity; and 2) reconstitution of in vivo impaired APC's function either by the adoptive transfer of ex vivo-activated APC (i.e. replacement strategy) or by a direct in vivo activation of less impaired DCs (such as Langerhans cells) by a simultaneous combination of the immunogen and appropriate cytokines or adjuvants. The ultimate goal of a successful therapeutic vaccine is to sustainably reduce the viral load of HIV-1-infected patients to as a low level as possible. This should protect them from disease progression, and thus reduce the requirement for harmful and expensive antiretroviral drugs. Moreover, sustainably reducing HIV viral load could minimize their risk of sexually transmitting the virus to healthy people (T. C. Quinn et al., Viral load and heterosexual transmission of human immunodeficiency virus type 1. Rakai Project Study Group. N Engl J Med 342, 921-9 (2000)).

A vaccine-induced protective immune response can differ substantially depending on the nature of the immunogen. Live-attenuated vaccines elicit both humoral and cellular immune responses, while killed virus vaccines and purified synthetic proteins preferentially elicit humoral (antibody) response. In a multi-center clinical trial aimed at triggering the cellular arm of the immune system, researchers were disappointed in that only 20% of 205 volunteers immunized with an experimental vaccine made of bacterial DNA containing HIV genes had a significant HIV-specific cellular immune response (although the DNA prime does work well in mouse experiments) (J. Cohen, AIDS vaccines. HIV dodges one-two punch. Science 305, 1545-7 (2004)). In this context, we (and others) recently discovered that inactivated whole HIV-1, in which the conformational structure of envelope protein gp120 is conserved (which is for example the case when the virus is treated by aldrithiol-2 [AT-2]), can be processed and presented by dendritic cells (DCs, the most potent APC) for inducing a potent HLA-I-restricted CTL response in vitro (W. Lu & J. M. Andrieu, In vitro HIV eradication by autologous CD8+ T cells expanded with inactivated-virus-pulsed dendritic cells. J Virol 75, 8949-56 (2001) and F. Buseyne et al., MHC-I-restricted presentation of HIV-1 virion antigens without viral replication. Nat Med 7, 344-9 (2001)). Several studies have also demonstrated that the adoptive transfer of autologous DCs loaded in vitro with inactivated whole HIV-1 induced protective antiviral immunity in hu-PBL-SCID mice (C. Lapenta et al., Potent immune response against HIV-1 and protection from virus challenge in hu-PBL-SCID mice immunized with inactivated virus-pulsed dendritic cells generated in the presence of IFN-alpha. J Exp Med 198, 361-7 (2003) and A. Yoshida et al., Induction of protective immune responses against R5 human immunodeficiency virus type 1 (HIV-1) infection in hu-PBL-SCID mice by intrasplenic immunization with HIV-1-pulsed dendritic cells: possible involvement of a novel factor of human CD4(+) T-cell origin. J Virol 77, 8719-28 (2003). We previously showed that a therapeutic vaccine made of inactivated whole simian immunodeficiency virus (SIV) strain mac251 (SIVmac251)-loaded DCs led, in the absence of any other antiviral therapy, to dramatic viral suppression in Chinese rhesus monkeys immunized two months been infection with SIVmac251 (W. Lu, X. Wu, Y. Lu, W. Guo & J. M. Andrieu, Therapeutic dendritic-cell vaccine for simian AIDS. Nat Med 9, 27-32 (2003)). The extensive global variability of HIV-1 argues against the concept of pharmaceutical use of a GMP-grade inactivated whole virus preparation as a universal therapeutic vaccine for HIV-1.

### SUMMARY OF THE INVENTION

This invention provides a vaccine comprising inactivated whole HIV of a specific subtype, optionally with an adjuvant. The vaccine can be used to treat individuals chronically infected with HIV by inducing a protective cellular immune response against the same HIV subtype used to produce the vaccine.

The invention also provides a pharmaceutical composition comprising a vaccine comprising inactivated whole HIV of a specific subtype and a pharmaceutically acceptable carrier.

The invention further provides the use of inactivated whole HIV of a specific subtype for producing a medicament for treating an individual chronically infected with HIV. The medicament can be used to treat individuals chronically infected with HIV by inducing a protective cellular immune response against the same HIV subtype used to produce the vaccine.

The invention still further provides a method of treating an individual chronically infected with HIV comprising administering a vaccine comprising inactivated whole HIV of a specific subtype to the individual, optionally with an adjuvant, so that a protective cellular immune response against the same HIV subtype used to produce the vaccine is induced in the individual. The inactivated HIV may be loaded ex vivo into antigen having immune cells (APCs), which are then administered to the individual.

### BRIEF DESCRIPTION OF THE DRAWINGS

FIG. 1 is a graph showing the cytotoxic T-lymphocyte (CTL) killing activity of vaccines of the invention.

FIG. 2 is a graph showing the effects of intra-dermal immunization with AT-2 inactivated SIV mac251 on the plasma viral load of chronically SIVmac251-infected macaques. The data represent the geometric mean of SIV RNA copies per milliliter of plasma before, and after immunization. P value represents the statistical analysis of paired data before, and at month 3 or 6 after the immunization by the Wilcoxon test.

### DETAILED DESCRIPTION

There are two major HIV groups (HIV-1 and HIV-2) and many subgroups because the HIV genome mutates constantly. The major difference between the groups and subgroups is in the viral envelope. HIV-1 is classified into main subgroup (M) and a 10th outlier subgroup (O), in which subgroup M is divided into nine subtypes (clades) designed A through J (Hu et al., JAMA 275:210-216 (1996) and Korber et al., Science 280:1868-1871 (1998)). The genetic variation seen in the HIV genome is the result of mutation, recombination, insertion, and deletion (Korber et al., Science 280:1868-1871 (1998)).

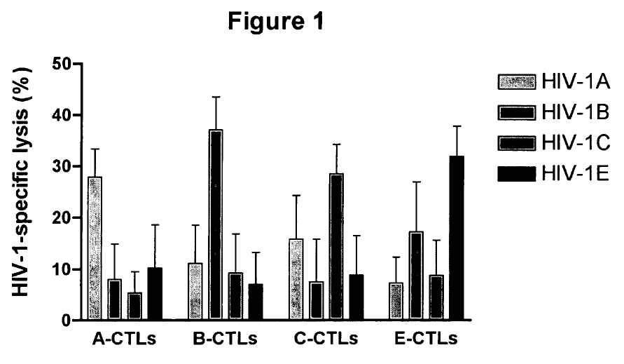

Vaccines against HIV, preferably HIV-1 subtypes like subtypes A, B, C, or E exhibited high CTL killing activity with subtype-matched viral strains while only low cross-subtype killing activity was observed. Although the cross-subtype killing activity was observed in individual cases, the CTL killing efficiency varied widely in the subtype-unmatched viral strains as compared to the subtype-matched viral strains (see Ex. 1 below and FIG. 1). Thus, we provide a subtype-specific therapeutic vaccine for HIV made with an inactivated whole HIV preparation of a given subtype. The vaccines can be used to treat an individual with a chronic HIV infection, wherein the vaccines induce a protective cellular immune response against the same HIV subtype used to produce the vaccine.

The term “vaccine” as used herein, refers to a composition that is administered to produce or artificially increase immunity to a particular disease.

Preferably, the vaccines are produced with an HIV subtype which is not autologous to the individual being treated. In fact, we have shown that unexpectedly a vaccine with an HIV subtype which is not autologous to the individual to be treated enable to significantly decrease the viral charge of the individual.

A chronic infection with any HIV subtype can be treated. For example, chronic infections with HIV virus are selected from one of the A, B, C, or E (and other) subtypes of the group M, as well HIV-2 group and HIV O subgroup.

In one aspect, the composition can comprise several inactivated HIV subtypes, e.g., two, three, four or more different inactivated HIV subtypes.

As used herein, “treating” an individual with a chronic HIV infection means that symptoms of HIV infection are prevented, reduced or inhibited; viral load (in particular, plasma viral load) is reduced after administering the vaccine; and/or an anti-HIV CTL response is induced in the individual. It is understood that “treating” an individual for chronic HIV infection does not require the complete eradication of HIV from the individual.

The term “individual with a chronic HIV infection” means an individual, which can be diagnosed as infected by HIV.

The term “inactivated whole HIV” means a complete HIV particle, which has been inactivated, and which is no longer infectious.

HIV of a specific subtype can be inactivated by any suitable technique known in the art, such as ultraviolet irradiation, heat or chemical treatment, like formaldehyde, paraformaldehyde, propiolactene or AT-2 treatment.

Preferably, HIV of a specific subtype is inactivated by exposing the HIV to a chemical treatment, and more preferably to AT-2 treatment. Unexpectedly, such an AT-2 inactivation enables maintaining the conformational structure of the HIV proteins, and the induction of real potent CTL response with non autologous.

The subtype-specific inactivated whole virus immunogens can be used either for ex vivo loading (also called “pulsing”) of antigen presenting immune cells (APCs), such as mature or immature dendritic cells (DCs) or Langerhans cells (LCs), to produce an APC-based therapeutic vaccine, or for a direct intradermic in vivo injection enabling the in vivo loading of LCs to produce a cell-free vaccine.

The cell-free vaccines can be administered by, for example, direct (preferably needle-free) delivery of the inactivated subtype-specific HIV (e.g., by an intradermic injector) to a patient's skin by methods determinable to one skilled in the art. Needle-free devices for intradermic vaccine administration are well known, and include, as an example, the devices described in U.S. Pat. No. 6,933,319 and WO 2004/101025, which are incorporated herein by reference. Vaccination through the skin (intradermal administration) is particularly advantageous, as the epidermis harbors large numbers of LCs. LCs are known to be the immature form of DCs which are located in close proximity to the most superficial layer of the skin, the stratum comeum. These LCs represent a network of immune cells that underlie 25% of the skin's surface area (R. C. Yu, D. C. Abrams, M. Alaibac & A. C. Chu, Morphological and quantitative analyses of normal epidermal Langerhans cells using confocal scanning laser microscopy. Br J Dermatol 131, 843-8 (1994)) and remain functionally intact during the early or asymptomatic phase of chronic HIV/SIV infection (M. I. Zimmer et al., Disrupted homeostasis of Langerhans cells and interdigitating dendritic cells in monkeys with AIDS. Blood 99, 2859-68 (2002)). We have shown that such an intradermic administration of the cell-free vaccines enable a significant decrease in SIV viral charge.

The APC-based vaccines of the invention can be administered by, for example, direct delivery of the APC loaded with inactivated subtype-specific HIV (e.g., by a subcutaneous injector) to patient by methods within the skill in the art.

The method may comprise a previous step of determining the subtype of HIV infecting the patient to be treated before administration of the composition. Such a determination can be done by well known methods like genotyping of specific area of HIV sequence by analysis of HIV present in a blood sample from the patient. Preferably, this HIV subtype determination step is followed by administration of APC-based or cell free vaccines comprising the same HIV subtype than those infecting the patient to be treated.

An individual may be treated with APC loaded with inactivated HIV of a specific subtype. The APC is first loaded with the inactivated HIV ex vivo, and the loaded APC is then administered to the patient by any suitable technique. Preferably, the loaded APC is injected subcutaneously, intradermally or intramuscularly into the individual, preferably by a subcutaneous injection. More preferably, the APC are obtained by a previous PBMC sampling from the individual to be treated to isolate the monocytes (CD14+), which are then transformed with known cytokines in immature and then mature dendritic cells. Such methods are well known, as an example, such a method is described in Example 1.

Unlike bacterial products, inactivated whole HIV alone are not as efficient for inducing maturation of DCs (W. Lu, X. Wu, Y. Lu, W. Guo & J. M. Andrieu, Therapeutic dendritic-cell vaccine for simian AIDS. Nat Med 9, 27-32 (2003)) to allow an efficient migration of these cells to the draining lymph nodes, where they execute their immunostimulatory function. Therefore, a class of potent molecules known as “adjuvants” is preferably combined with the inactivated whole HIV for triggering optimal maturation of immune cells such as LCs, thereby allowing an efficient cellular immune response against HIV-1 to be generated.

The term “adjuvant” refers to a substance added to a vaccine to improve the immune response.

Suitable adjuvants include, but are not limited to, at least one of complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, conventional bacterial products (such as cholera toxin, heat-labile enterotoxin, attenuated or killed BCG (bacille Calmette-Guerin) and Corynebacterium parvum, or BCG derived proteins), biochemical molecules (such as TNF-alpha, IL-1-beta, IL-6, PGE₂, or CD40L), or oligodeoxynucleotides containing a CpG motif. Examples of materials suitable for use in vaccine compositions are disclosed, e.g., in Osol, A., ed., Remington's Pharmaceutical Sciences, Mack Publishing Co., Easton, Pa. (1980), pp. 1324-1341, which reference is entirely incorporated herein by reference.

Dendritic cells have a pivotal role in monocytes differentiation regulation, and more especially in the regulation of CD4-Th1 profile versus CD4-Th2 profile differentiation. Preferably, the cell-free vaccine composition comprises adjuvants able to stimulate dendritic cells to inhibit cellular differentiation in CD4-Th2 profile, which is often induced in chronic infection, and simultaneously able to stimulate dendritic cells to activate cellular differentiation in CD4-Th1 profile. Such adjuvants are well known and include bacterial products (such as some BCG-derived proteins like Ag85B) or chemical compounds like compounds with anti-COX activity, and more especially with anti-COX2 activity (such as VIOX®, CELEBREX® or RIBAVERIN®).

The vaccines can be formulated into pharmaceutical compositions (also called “medicaments”) for treating an individual chronically infected with HIV. Pharmaceutical compositions are preferably sterile and pyrogen free, and also comprise a pharmaceutically acceptable carrier. Suitable pharmaceutically acceptable carriers include, but are not limited to, at least one of water, saline solutions (e.g., physiological saline), viscosity adjusters and other conventional pharmaceutical excipients and/or additives used in the formulation of pharmaceutical compositions for use in humans. Suitable pharmaceutical excipients include, but are not limited to, at least one of stabilizers, antioxidants, osmolality adjusting agents, buffers, and pH adjusting agents. Suitable additives include, but are not limited to, at least one of physiologically biocompatible buffers (e.g., tromethamine hydrochloride and the like), chelants (e.g., DTPA, DTPA-bisamide and the like) or calcium chelate complexes (e.g., calcium DTPA, CaNaDTPA-bisamide and the like), or, optionally, additions of calcium or sodium salts (e.g., calcium chloride, calcium ascorbate, calcium gluconate, calcium lactate and the like). Formulation of pharmaceutical compositions are within the skill in the art, for example, as described in Remington's Pharmaceutical Science, 17th ed., Mack Publishing Company, Easton, Pa. (1985), the entire disclosure of which is herein incorporated by reference.

A typical regimen for treating an individual chronically infected with HIV which can be alleviated by a cellular immune response by active therapy, comprises administration of an effective amount of a vaccine composition as described above, administered as a single treatment, or repeated as enhancing or booster dosages, over a period up to and including one week to about 24 months.

An “effective amount” of a vaccine composition is one which is sufficient to achieve a desired biological effect, in this case at least one of cellular or humoral immune response to HIV, preferably one cellular immune response. It is understood that the effective dosage will be dependent upon the age, sex, health, and weight of the recipient, kind of concurrent treatment, if any, frequency of treatment, and the nature of the effect desired. The ranges of effective doses provided below are not intended to limit the invention and represent preferred dose ranges. However, the most preferred dosage will be tailored to the individual subject, as is understood and determinable by one of skill in the art, without undue experimentation. See, e.g., Berkow (1987), infra, Goodman (1990), infra, Avery (1987), infra, Ebadi, Pharmacology, Little, Brown and Co., Boston, Mass. (1985), and Katsung (1992), infra, which references and references cited therein, are entirely incorporated herein by reference.

Generally speaking, the dosage for a human adult will be from about 10⁶-10¹⁴ inactivated whole HIV particles per dose, with 10⁸-10¹² preferred. Whatever dosage is used, it should be a safe and effective amount as determined by known methods, as also described herein.

The invention will now be illustrated by the following non-limiting examples.

### EXAMPLES

### Example 1

Methods

Virus and Cell Samples

HIV-1 strains were obtained by CD8-depleted peripheral blood mononuclear cell (PBMC) culture from patients infected with HIV-1 subtypes A (n=10), B (n=10), C (n=10), or E (n=10). These HIV-1 subtype (A, B, C, or E) strains were then inactivated by AT-2 (Sigma, St Louis, Mo.) as described in W. Lu & J. M. Andrieu, (In vitro HIV eradication by autologous CD8+ T cells expanded with inactivated-virus-pulsed dendritic cells. J Virol 75, 8949-56 (2001)), the entire disclosure of which is herein incorporated by reference. Monocyte-derived DCs were prepared from each patient by a standardized 7-day culture (W. Lu, X. Wu, Y. Lu, W. Guo & J. M. Andrieu, Therapeuti dendritic-cell vaccine for simian AIDS. Nat Med 9, 27-32 (2003)) under GMP conditions. Briefly, freshly collected PBMCs were subjected to plastic adherence at a density of 10⁶ cells/cm² in the presence of 0.5% of clinical-use human serum albumin (LFB, Les Ulis, France). After 2-hour incubation at 37° C. in 5% CO₂, non-adherent cells were removed by rinsing with sterile PBS buffer. Adherent cells were then cultured for 5 days in a complete medium containing clinical-grade CellGro DC medium (CellGenix, Freiburg, Germany) supplemented with 2000 U/ml GM-CSF (Schering-Plough, Brinny, Ireland) and 50 ng/ml clinical-grade IL-4 (CellGenix). At day 5, DCs were exposed to AT-2-inactivated autologous virus (10⁹ viral particles/ml) at 37° C. for 2 h. After 2 washes to remove non-bound inactivated virus, cells were cultured for 2 additional days in the complete medium supplemented with clinical-grade cytokines IL-1β (10 ng/ml) (CellGenix), IL-6 (100 ng/ml) (CellGenix), and TNF-α (50 ng/ml) (CellGenix). At day 7, quality control (QC) of DCs was performed by flow cytometry (W. Lu, X. Wu, Y. Lu, W. Guo & J. M. Andrieu, Therapeuti dendritic-cell vaccine for simian AIDS. Nat Med 9, 27-32 (2003)), the entire disclosure of which is herein incorporated by reference. QC-approved viable DCs were then used for expanding autologous virus-specific CTLs by a coculture protocol as described in W. Lu & J. M. Andrieu (In vitro HIV eradication by autologous CD8+ T cells expanded with inactivated-virus-pulsed dentritic cells. J Virol 75, 8949-54 (2001)), supra.

Cytotoxic Assay

DCs were pulsed with AT-HIV-1 (10⁹/ml) for 90 min then labeled with CFSE (Molecular Probes, PoortGebouw, The Netherlands) (10 nM) for 15 min and washed twice. Non-pulsed DCs were labeled with CFSE as specificity control. HIV-1 subtype strain-specific CTLs were plated into Micro Tubes-Bulk (Bio-Rad, Hercules, Calif.) in the presence of CFSE-labeled subtype-matched or unmatched AT-2-HIV-1-pulsed dendritic cells (DCs) at an E:T ratio of 10:1 for 4 h at 37° C. At the end of the incubation, 10 μl of propidium iodide (PI, Sigma) (20 μg/ml) were added to each tube. Target cytolysis was analyzed on a FACSCalibur (BD Immunocytometry System, San Jose, Calif.). Virus-specific cytolytic activity was determined by calculating the percentage of CFSE/PI-staining DCs after subtraction of the non-specific CFSE/PI-staining of non-pulsed DCs.

Statistical Analysis

Impaired data between HIV-1 subtype-matched and subtype-unmatched cell-killing activities were compared by the Mann-Whitney test.

Results

Vaccines specific to HIV-1 subtypes A, B, C, or E exhibited high in vitro CTL killing activity (28-37%) with subtype-matched viral strains (n=10), while only low (5-17%) cross-subtype killing activity was observed (P<0.001). Although the cross-subtype killing activity was observed in individual cases, the CTL killing efficiency varied widely in the subtype-unmatched viral strains (SD/mean>50%) as compared to the subtype-matched viral strains (SD/mean<20%) (P<0.001) (FIG. 1). These findings indicate that a pharmaceutical subtype-specific therapeutic vaccine for HIV-1 can be made by a GMP-grade inactivated whole virus preparation.

### Example 2

Individuals chronically infected with HIV are identified, and the HIV subtype with which the individual is infected is determined. Plasma viral load is determined for each individual prior to treatment with a vaccine of the invention. HIV of the same subtype as the subtype with which the individuals are infected is obtained, cultured and inactivated with AT-2 as described above in Example 1.

Inactivated HIV subtypes are administered to one group chronically infected individuals by needleless intradermal delivery, and plasma viral load is measured. It is expected that plasma viral load in the chronically infected individuals will be significantly reduced upon administration of the vaccines.

Another group of chronically infected individuals are administered dendritic cells which have been loaded with HIV inactivated with AT-2 as described above in Example 1. The HIV-loaded dendritic cells are administered to the individuals. It is expected that plasma viral load in the chronically infected individuals will be significantly reduced upon administration of the loaded dendritic cells.

### Example 3

40 chronically (>1 year) SIVmac251-infected macaques with a plasma viral load>1000 copies/ml were randomized to either receive a monthly intra-dermal injection (25 cm² on the back) of the SIVac LA2.1 (2.5 ml 0.9% NaCl solution containing 10¹⁰ AT-2-inactivated SIVmac251) for 5 months using an automated injecting pistol (AKRA DERMOJET, Pau, France) (100 μl/cm²/injection) (n=20); or receive a monthly intra-dermal injection of placebo (2.5 ml 0.9% NaCl solution alone) for 5 months (n=20). Plasma samples were collected from baseline and every month thereafter up to 6 months and stored at −80° C. until use. Finally, the plasma SIV RNA load was measured by a quantitative RT-PCR assay (MUPROVAMA).

Results

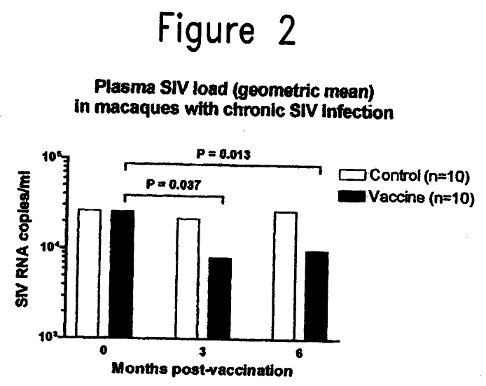

Cell-free vaccines specific to SIV subtype induce nearly 30% reduction (FIG. 2) in blood viral charge at 3 months (P=0.037), and at 6 months (P=0.013). These findings indicate that a pharmaceutical subtype-specific therapeutic vaccine for SIV can be made by a GMP-grade inactivated whole virus preparation.

### Example 4

140 chronically (>1 year) SIVmac251-infected macaques with a plasma viral load>1000 copies/ml were randomized to either receive a monthly intra-dermal injection (25 cm² on the back) of the SIVac LA2.1 (2.5 ml 0.9% NaCl solution containing 10¹⁰ AT-2-inactivated SIVmac251) with different adjuvants (10⁵ UFC of attenuated or heat-killed BCG, BCG-derived recombinant Ag85B) for 5 months using an automated injecting pistol (AKRA DERMOJET, Pau, France) (100 μl/cm²/injection) with or without a daily oral administration of 200 mg of CELEBREX® for 4 weeks (n=20 for each group); or receive a monthly intra-dermal injection of placebo (2.5 ml 0.9% NaCl solution alone) for 5 months (n=20). Plasma samples were collected from baseline and every month thereafter up to 6 months and stored at −80° C. until use. Finally, the plasma SIV RNA load was measured by a quantitative RT-PCR assay (MUPROVAMA).

All documents referenced in this application, including those listed above, are herein incorporated by reference in their entirety. A variety of modifications to the embodiments described above will be apparent to those skilled in the art from the disclosure provided herein. Thus, the invention may be embodied in other specific forms without departing from the spirit or essential attributes thereof.

We claim:

1. A composition comprising inactivated whole HIV of a specific subtype.

2. The composition of claim 1, further comprising a pharmaceutically acceptable carrier.

3. The composition of claim 1, wherein said inactivated whole HIV subtype is non autologous.

4. The composition of claims 1, wherein the whole inactivated HIV of a specific subtype is selected from group M subtypes, HIV-2 group and HIV O subgroup.

5. The composition of claim 4, wherein the group M subtype is A, B, C or E.

6. The composition of claim 1, wherein said inactivated whole HIV has been inactivated by chemical treatment.

7. The composition of claim 6, wherein said inactivated whole HIV has been inactivated by aldithriol-2 (AT-2) treatment.

8. The composition of claim 7, further comprising an adjuvant.

9. The composition of claim 8, wherein the adjuvant stimulates maturation of dendritic cells.

10. The composition of claim 8, wherein the adjuvant able to stimulate dendritic cells in order to inhibit cellular differentiation in CD4-Th2 profile.

11. The composition of claim 10, wherein the adjuvant able to stimulate dendritic cells in order to activate cellular differentiation in CD4-Th1 profile.

12. The composition of claim 1, wherein said inactivated whole HIV is presented by antigen presenting immune cells (APCs).

13. The composition of claim 12 wherein the APCs are selected from the group of Langerhans cells (LCs), mature and immature dendritic cells (DCs).

14. A method of treating an individual chronically infected with HIV, comprising administering a vaccine comprising whole, inactivated HIV of a specific subtype to the individual, wherein a protective cellular immune response against the same HIV subtype used to produce the vaccine is induced in the individual.

15. The method of claim 14, further comprising administering an adjuvant to the individual.

16. The method of claim 15, wherein the adjuvant stimulates maturation of dendritic cells.

17. The method of claim 16, wherein the dendritic cell is a Langerhans cell.

18. The medicament of claim 16, wherein the adjuvant able to stimulate dendritic cells to inhibit cellular differentiation in CD4-Th2 profile.

19. The composition of claim 16, wherein the adjuvant able to stimulate dendritic cells to activate cellular differentiation in CD4-Th1 profile.

20. The method of claim 14, wherein the inactivated whole HIV of a specific subtype is at least one selected from group M subtypes, HIV-2 group and HIV O subgroup.

21. The method of claim 20, wherein the group M subtype is A, B, C or E.

22. The method of claim 14, wherein the inactivated whole HIV of a specific subtype has been inactivated by chemical treatment.

23. The method of claim 22, wherein the inactivated whole HIV has been inactivated by aldithriol-2 (AT-2) treatment.

24. The method of claim 14, wherein the inactivated whole HIV is administered by intradermal delivery.

25. The method of claim 24, wherein the intradermal delivery is by direct needle-free delivery.

26. The method of claim 14, wherein the inactivated whole HIV of a specific subtype is not autologous to the individual being treated for chronic HIV infection.

27. The method of claim 14, comprising a first step of determining the subtype of the HIV infecting the individual to be treated.

28. The method of claim 27, wherein the administrated inactivated HIV and the HIV infected the individual to be treated have the same subtype.

29. A method of treating an individual chronically infected with HIV, comprising:
1) loading an antigen presenting cell ex vivo with an inactivated whole HIV of a specific subtype; and
2) administering the loaded antigen presenting cells to the individual to thereby induce a protective cellular immune response against the same HIV subtype used to produce the vaccine.

30. The method of claim 29, wherein the antigen presenting cell is a dendritic cell.

31. The method of claim 29, wherein the inactivated whole HIV of a specific subtype is at least one selected from group M subtypes, HIV-2 group and HIV O subgroup.

32. The method of claim 31, wherein the group M subtype is A, B, C or E.

33. The method of claim 29, wherein the inactivated whole HIV is administered by subcutaneous delivery.

34. The method of claim 29, comprising a first step of determining the subtype of the HIV infecting the individual to be treated.

35. The method of claim 34, wherein the administrated inactivated HIV and the HIV infected the individual to be treated have the same subtype.
